(19) United States
(12) Patent Application Publication
(10) Pub. No.: US 2019/0370960 A1
(43) Pub. Date: Dec. 5, 2019

(54) CROSS-STAINING AND MULTI-BIOMARKER METHOD FOR ASSISTING IN CANCER DIAGNOSIS

(71) Applicant: National Taiwan University of Science and Technology, Taipei City (TW)

(72) Inventors: CHING-WEI WANG, Taipei City (TW); YEN-LIN CHEN, Taipei City (TW)

(21) Appl. No.: 16/177,441

(22) Filed: Nov. 1, 2018

(30) Foreign Application Priority Data
May 30, 2018 (TW) 107118500

(51) Int. Cl. G06T 7/00; G01N 1/30; G01N 33/58

(52) U.S. Cl. CPC G06T 7/0012; G01N 33/582; G01N 1/30

(57) ABSTRACT

Disclosures of the present invention describe a cross-staining and multi-biomarker method for assisting in cancer diagnosis. The method is configured to firstly divide a plurality of image frames of tissue slices to a group of H&E-stained slide images and a group of IHC-stained slide images. Subsequently, an image registration and fusion process is applied to at least two cross-stained slide images consisting of at least one H&E-stained slide image and at least one IHC-stained slide image, thereby producing a plurality of cross-stained slide images. Consequently, by applying a carcinoma identifying and quantifying analysis to the cross-stained slide images, the type of cancerous lesions contained by the tested tissue sample can be diagnosed effectively and accurately, without any human-made judgements.

### BACKGROUND OF THE INVENTION

### 1. Field of the Invention

The present invention relates to the field of pathological image diagnosis technologies, and more particularly to a cross-staining and multi-biomarker method for assisting in cancer diagnosis.

### 2. Description of the Prior Art

Breast cancer most commonly develops in cells from the lining of milk ducts and the lobules that supply the ducts with milk. Doctors should know that breast cancer occurs when some breast cells begin to grow abnormally. These abnormally-growing cells may further spread (metastasize) through breast to lymph nodes. Breast cancer is mainly classified into three groups (types) of atypical ductal hyperplasia (ADH), ductal carcinoma in situ (DCIS) and basal-like Breast carcinoma (BC). To fully treat the breast cancer, it needs to firstly find some differences and/or relationships between normal cells, ADH cells, DCIS cells, and BC cells from tissue slice(s), and then doctors are able to design or plan at least one proper therapy for curing the breast cancer.

FIG. 1 shows a flowchart diagram for describing a treatment method of breast cancer. In step S1′ of the treatment method, mammography or breast sonography is conducted in order to facilitate an attending physician able to identify whether a patient's breast contains abnormal tumors or lesions or not, through primary image interpretation. When the primary image interpretation reports that the breast is at a normal condition, the patient merely needs to have regular appointments with her attending physician for tracking the breast's condition (step S2a′). However, in the case of the fact that the primary image interpretation reveals that the breast contains abnormal tumors or lesions, the treatment method subsequently proceeds to step S2′, such that a core needle biopsy is applied to the patient's breast. Next, the attending physician can categorize the lesion as ADH or DCIS based on tissue slice analysis report under the execution of step S3′. When the breast cancer is eventually categorized as ADH, it is necessary for the patient to subsequently receive a surgical treatment in order to remove the cancerous lesions (step S4′). It is worth further explaining that, pathological analysis for the removed lesions is further conducted in step S4′. In the case of the fact that neoplastic transformation found from a portion of the removed lesions is categorized as ADH, the patient merely needs to have regular appointments with the attending physician for continuously tracking the her breast's condition (step S7′).

On the contrary, once the pathological analysis result indicates that DCIS have become invasive and spread in all of the sample of the cancerous lesion, MRI equipment will be further used for determining whether the lesion is a multiple carcinoma or not (step S5′). Subsequently, the treatment method proceeds to step S6′. During the execution of step S6′, surgical treatment (mastectomy) is applied to the patient again for removing a portion of breast when the cancerous lesion is identified as a multiple carcinoma in step S5′. However, all of the patient's breast must be removed in the case of the tumor being categorized as a multiple carcinoma. Consequently, step 7′ is configured to apply other postoperative treatment(s) to the patient, including applying a breast reconstruction to the patient already been removed all of her breast. Moreover, it is noted that, radiation therapy or hormone therapy is still necessary for the patient already been removed a portion of breast.

From FIG. 1, it is understood that accuracy of the pathological analysis based on at least one tissue slice dominates the design and plan of the breast cancer treating therapy made by doctors. Accordingly, image registration, a process of transforming different sets of data into one coordinate system, is developed and potentially an enabling technology for the effective and efficient use of many image guided diagnostic and treatment procedures, which rely on multimodality image fusion or serial image comparison. For instance, U.S. Pat. No. 9,818,190 B2 discloses a whole slide image registration and cross-image annotation system. The disclosed system installed with computer software products for aligning whole slide digital images on a common grid and transferring annotations from one aligned image to another aligned image on the basis of matching tissue structure.

From U.S. Pat. No. 9,818,190 B2, it is understood that images of tissue slices already been applied with hematoxylin and eosin (H&E) staining treatment are classified as source images, and images of tissue slices already been applied with immunohistochemistry (IHC) staining treatment are classified as target images. Particularly, after one of the source image has labeled with user-marked annotations, side-by-side viewing of matched Field of Views (FOVs) from the source image and at least one target image corresponding to the source image is provided by the system, so as to enable a user (i.e., the doctor) to compare the user-marked FOV with the algorithm-retrieved FOV in the corresponding target image(s). Briefly speaking, the disclosed system enables doctor to select images for alignment (registration) in a set of images obtained from a tissue section of a single patient, wherein each image in the set may have been made using different staining ways.

However, it is a pity that the disclosed whole slide image registration and cross-image annotation system is unable to automatically classify abnormal tumors, cancerous lesions, or normal tissues based on IHC-stained slide images and H&E-stained slide images made from tissue slices. Accordingly, the inventors of the present application have made great efforts to make inventive research thereon and eventually provided a cross-staining and multi-biomarker method for assisting in cancer diagnosis.

### SUMMARY OF THE INVENTION

The primary objective of the present invention is to provide a cross-staining and multi-biomarker method for assisting in cancer diagnosis, wherein the method is configured to of firstly divide a plurality of image frames of tissue slices to a group of H&E-stained slide images and a group of IHC-stained slide images. Subsequently, an image registration and fusion process is applied to at least two cross-stained slide images consisting of at least one H&E-stained slide image and at least one IHC-stained slide image, thereby producing a plurality of cross-stained slide images. Consequently, by applying a carcinoma identifying and quantifying analysis to the plurality of cross-stained slide images based on a particularly-designed biomarker expression recognizing flow, various types of cancerous lesions formed in the tissue sample can be effectively detected and eventually diagnosed. Besides, an enrichment ratio of each of the diagnosed cancerous lesions can also be simultaneously calculated. Therefore, it is extrapolated that all types of the abnormal tumor cells or cancerous lesions contained by the tissue sample can be better diagnosed under the implementation of this novel method, without any human-made judgements.

In order to achieve the primary objective of the present invention, the inventor of the present invention provides one embodiment for the cross-staining and multi-biomarker method for assisting in cancer diagnosis, comprising following steps:
(1) preparing a tissue sample containing at least one breast milk duct, and then processing the tissue sample to a plurality of tissue slices;
(2) dividing the plurality of tissue slices into a H&E-stained tissue slice group and a IHC-stained tissue slice group, wherein the IHC-stained tissue slice group comprising a first tissue slice group with fluorescent labeled E-cadherin, a second tissue slice group with fluorescent labeled tumor protein p63, a third tissue slice group with fluorescent labeled cytokeratin (CK) 14, and a fourth tissue slice group with fluorescent labeled CK 5/6;
(3) respectively applying a H&E staining treatment and an IHC staining treatment to the tissue slices in the H&E-stained tissue slice group and the tissue slices in the IHC-stained tissue slice group, so as to obtain a plurality of H&E-stained slices and a plurality of IHC-stained slices;
(4) processing the H&E-stained slices to a plurality of H&E-stained slide images, and also processing the IHC-stained slices to a plurality of IHC-stained slide images;
(5) applying an image registration and fusion process to at least two cross-stained slide images consisting of at least one H&E-stained slide image and at least one IHC-stained slide image;
(6) repeating the step (5) until all of the H&E-stained slide images and the IHC-stained slide images have been treated with the image registration and fusion process, thereby producing a plurality of cross-stained slide images; and
(7) applying a carcinoma identifying analysis to the plurality of cross-stained slide images, so as to complete the identification of at least one type of cancerous lesion and/or lesion by carrying out image interpretations of the cross-stained slide images.

### BRIEF DESCRIPTION OF THE DRAWINGS

The invention as well as a preferred mode of use and advantages thereof will be best understood by referring to the following detailed description of an illustrative embodiment in conjunction with the accompanying drawings, wherein:

FIG. 1 shows a flowchart diagram for describing a treatment method of breast cancer;

FIG. 2A and FIG. 2B show flowchart diagrams for describing a cross-staining and multi-biomarker method for assisting in cancer diagnosis according to the present invention;

FIG. 3A and FIG. 3B show schematic diagrams for depicting the manufacturing flow of cross-stained slide images;

FIG. 4A, FIG. 4B and FIG. 4C show schematic diagrams for depicting an image registration and fusion process;

FIG. 5A, FIG. 5B, FIG. 5C, and FIG. 5D show flowchart diagrams for describing detail execution steps of step S7 of the cross-staining and multi-biomarker method;

FIG. 6 shows a cross-sectional view of a breast milk duct;

FIG. 7 shows four frames of cross-stained slide images and one frame of H&E-stained slide image;

FIG. 8 shows four frames of cross-stained slide images and one frame of H&E-stained slide image;

FIG. 9 shows four frames of cross-stained slide images and one frame of H&E-stained slide image;

FIG. 10 shows four frames of cross-stained slide images and one frame of H&E-stained slide image; and

FIG. 11 shows four frames of cross-stained slide images and one frame of H&E-stained slide image.

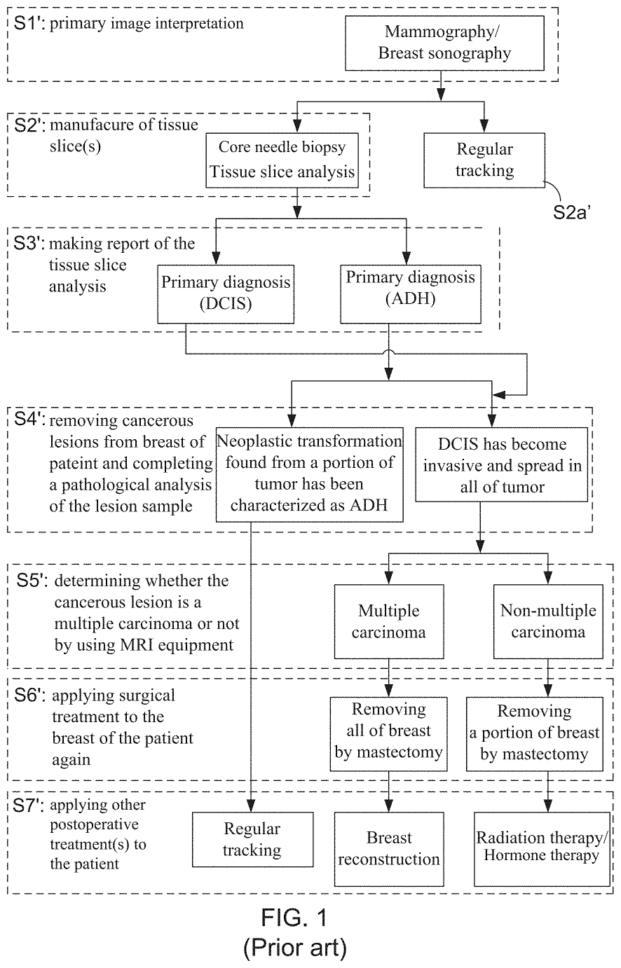

### DETAILED DESCRIPTION OF THE PREFERRED EMBODIMENTS

To more clearly describe a cross-staining and multi-biomarker method for assisting in cancer diagnosis according to the present invention, embodiments of the present invention will be described in detail with reference to the attached drawings hereinafter.

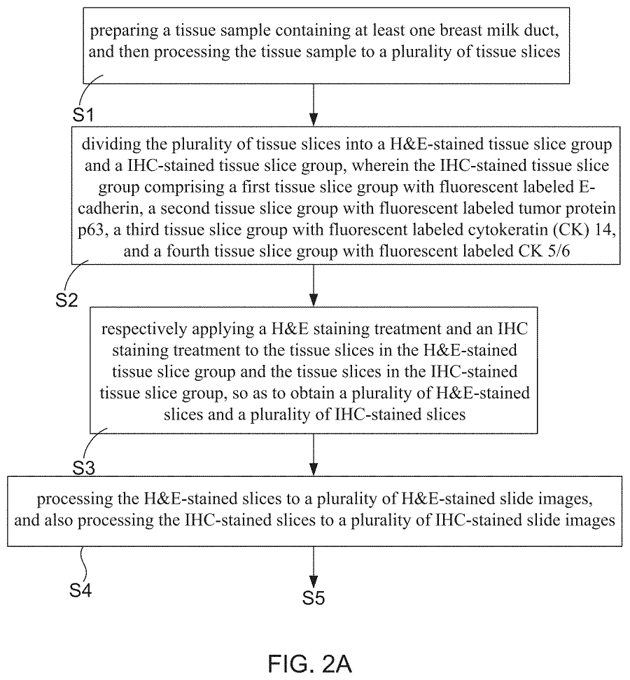

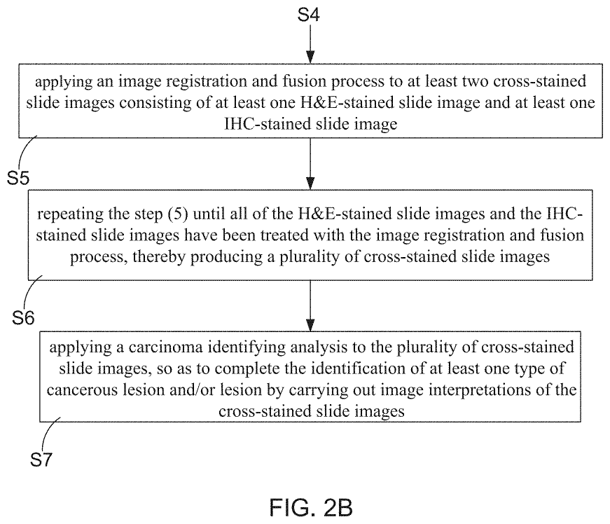

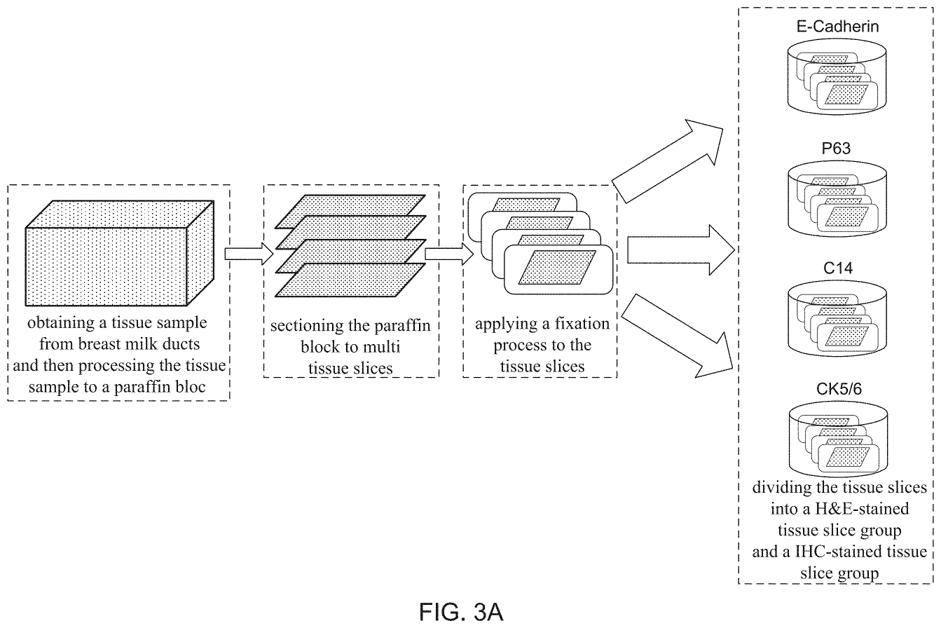

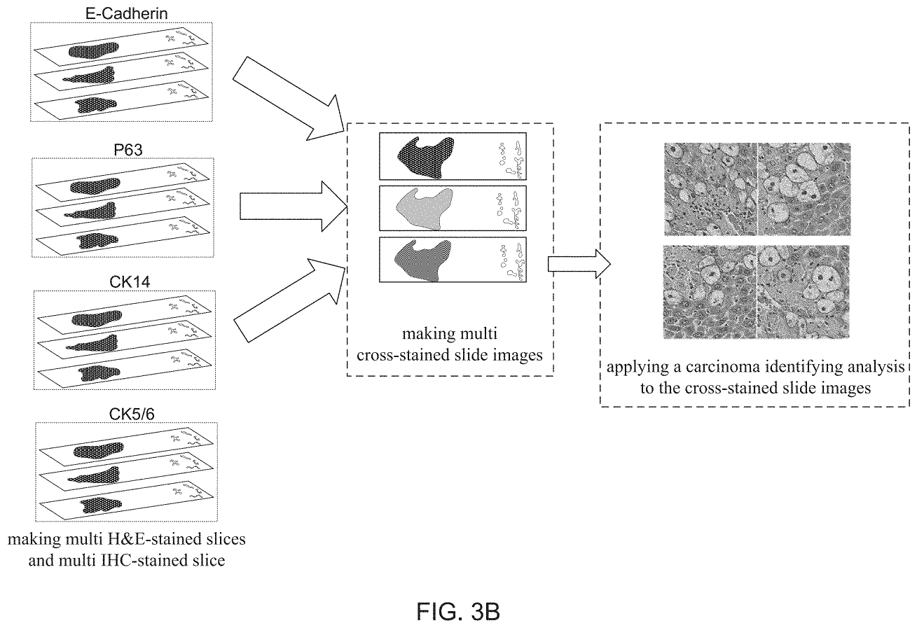

With reference to FIG. 2A and FIG. 2B, there are provided flowchart diagrams for describing a cross-staining and multi-biomarker method for assisting in cancer diagnosis according to the present invention. Moreover, please simultaneously refer to FIG. 3A and FIG. 3B, which illustrate schematic diagrams for depicting the manufacturing flow of cross-stained slide images. The cross-staining and multi-biomarker method of the present invention firstly proceeds to step S1, so as to prepare a tissue sample containing at least one breast milk duct and then further process the tissue sample to a plurality of tissue slices. From FIG. 3A, it is understood that, the tissue sample obtained from milk ducts is firstly processed to a paraffin block. Subsequently, after sectioning the paraffin block to multi tissue slices, a fixation process is then applied to the tissue slices.

In step S2, the plurality of tissue slices is divided into a H&E-stained tissue slice group and a IHC-stained tissue slice group. For instance, in order to carry out a hematoxylin and eosin (H&E) staining process and an immunohistochemistry (IHC) staining process, doctors certainly select a plurality of protein markers from the tissue slices by the use of a proteomics-based method. In exemplary case, the protein marker can be E-cadherin, tumor protein p63, smooth muscle protein (SMA), high molecular weight cytokeratin (HMCK), CK 14, CH 7, CK 5/6, or CK 8/18. Briefly speaking, the IHC-stained tissue slice group may at least comprises a first tissue slice group with fluorescent labeled E-cadherin, a second tissue slice group with fluorescent labeled tumor protein p63, a third tissue slice group with fluorescent labeled cytokeratin (CK) 14, and a fourth tissue slice group with fluorescent labeled CK 5/6.

The method is subsequently proceeded to step S3, so as to respectively apply a H&E staining treatment and an IHC staining treatment to the tissue slices in the H&E-stained tissue slice group and the tissue slices in the IHC-stained tissue slice group, thereby obtaining a plurality of H&E-stained slices and a plurality of IHC-stained slices. As FIG. 3 shows, the IHC-stained slices are classified to four tissue slice groups in order to assist doctors in breast cancer diagnosis, including a first tissue slice group with fluorescent labeled E-cadherin, a second tissue slice group with fluorescent labeled tumor protein p63, a third tissue slice group with fluorescent labeled CK 14, and a fourth tissue slice group with fluorescent labeled CK 5/6. Moreover, as FIG. 2A and FIG. 3B show, the method subsequently proceeds to step S4, such that the H&E-stained slices and the IHC-stained slices are further processed to a plurality of H&E-stained slide images and a plurality of IHC-stained slide images, respectively.

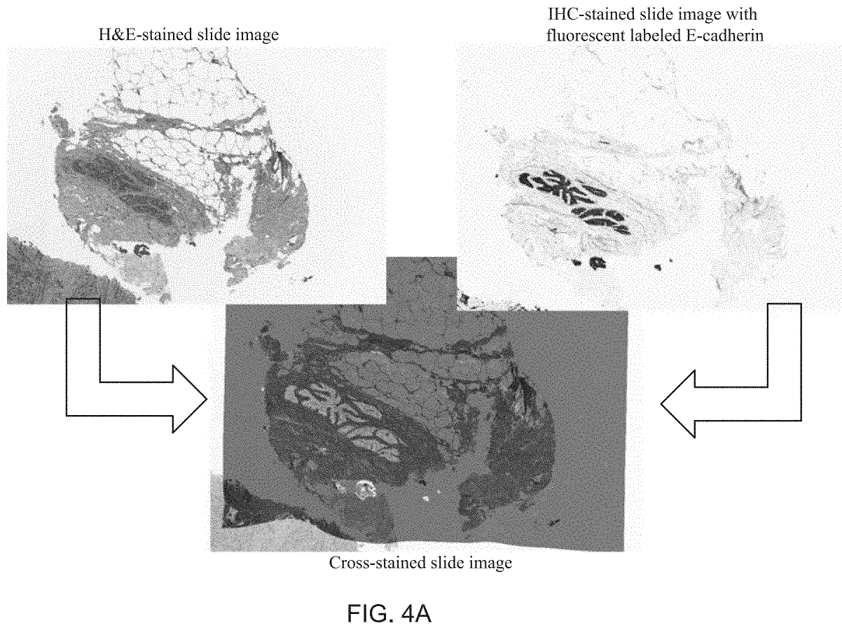

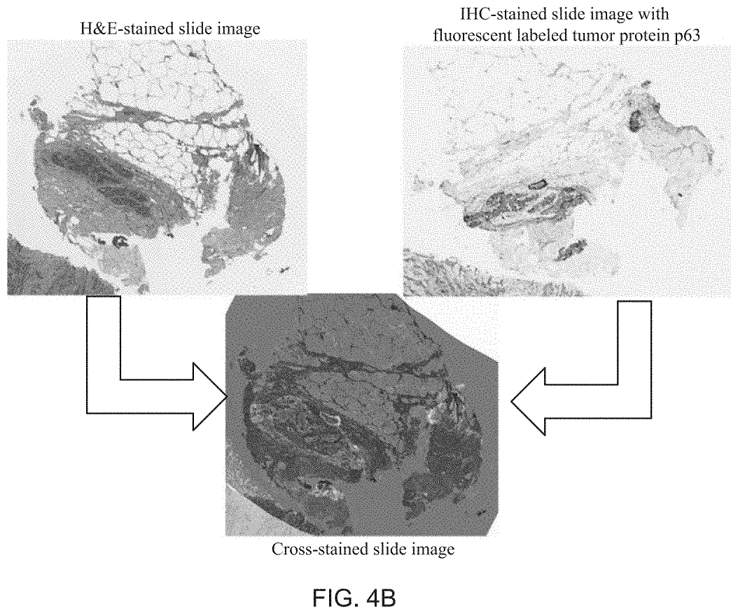

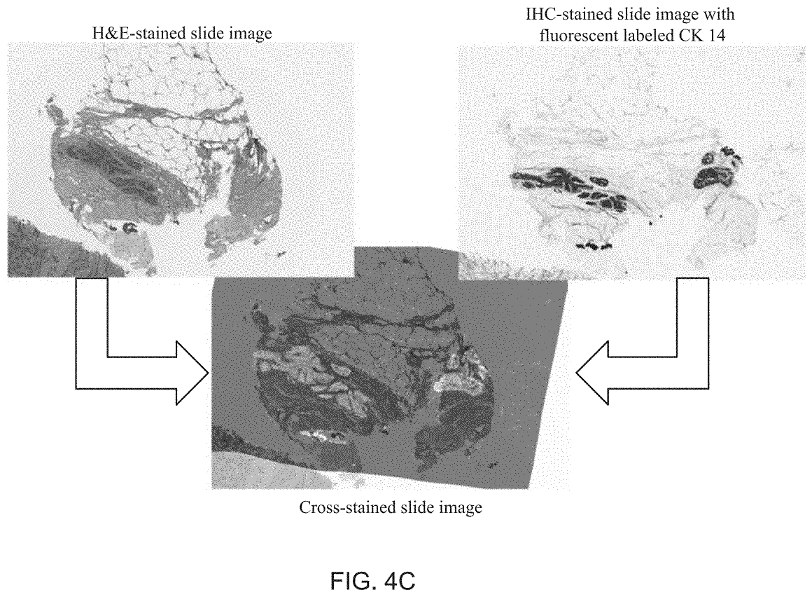

Furthermore, in steps S5, an image registration and fusion process is applied to at least two cross-stained slide images consisting of at least one H&E-stained slide image and at least one IHC-stained slide image. Moreover, step S6 is executed for repeating the step S5 until all of the H&E-stained slide images and the IHC-stained slide images have been treated with the image registration and fusion process, thereby producing a plurality of cross-stained slide images. FIG. 4A, FIG. 4B and FIG. 4C particularly illustrate schematic diagrams for depicting the image registration and fusion process. From FIG. 3B, FIG. 4A, FIG. 4B and FIG. 4C, it is understood that, a frame of IHC-stained slide image with fluorescent labeled E-cadherin, a frame of IHC-stained slide image with fluorescent labeled tumor protein p63, a frame of IHC-stained slide image with fluorescent labeled CK 14, and a frame of H&E-stained slide image are chosen to be further treated with the image registration and fusion process. It is known that, image alignment or registration processing technologies has been well developed and already widely applied between at least one target image and a corresponding source image thereof, thereby assisting in lesion identification through the image interpretation of tissue slide images. Commercial or conventional image alignment or registration processing technologies comprises: Least squares, UnwarpJ, UnwarpJ, Elastic, CwR, CLAHE+bunwarpJ, and TrakEM2. However, it is worth emphasizing that, the image registration and fusion processing technology adopted for being used in the step S5 does come from literature 1. Herein, Literature 1 is written by Wang et. al with title of “Robust image registration of biological microscopic images”, and is published on Nature-Scientific Reports 4: 6050 (SCI, JCR 2015 (7/63) in MULTIDISCIPLINARY SCIENCES, IF=5.228).

As FIG. 4A shows, a first cross-stained slide image constituted by the IHC-stained slide image with fluorescent labeled E-cadherin and the H&E-stained slide image is produced after completing the image registration and fusion process. Moreover, from FIG. 4B and FIG. 4C, it is understood that, a second cross-stained slide image is constituted by the H&E-stained slide image and the IHC-stained slide image with fluorescent labeled tumor protein p63, and a third cross-stained slide image is constituted by the H&E-stained slide image and the IHC-stained slide image with fluorescent labeled CK 14. Although there is no related diagram describing or depicting a fourth cross-stained slide image, it is extrapolated that the above-mentioned IHC-stained slide image with fluorescent labeled CK 5/6 is prepared for forming fourth cross-stained slide image with the H&E-stained slide image under the execution of the image registration and fusion process.

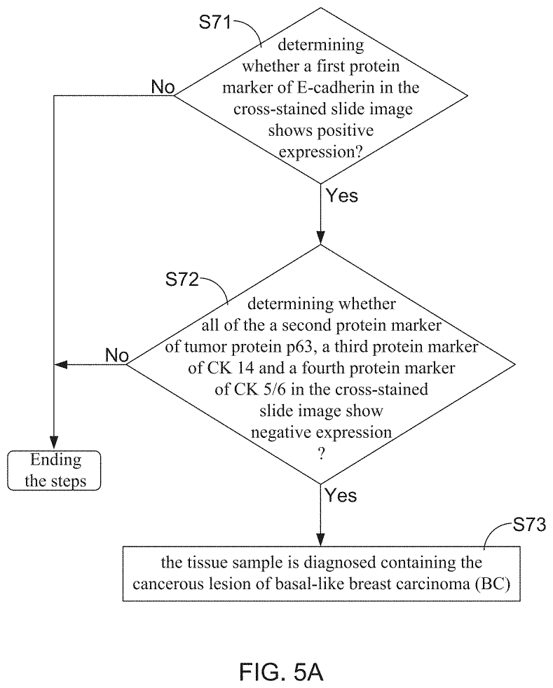

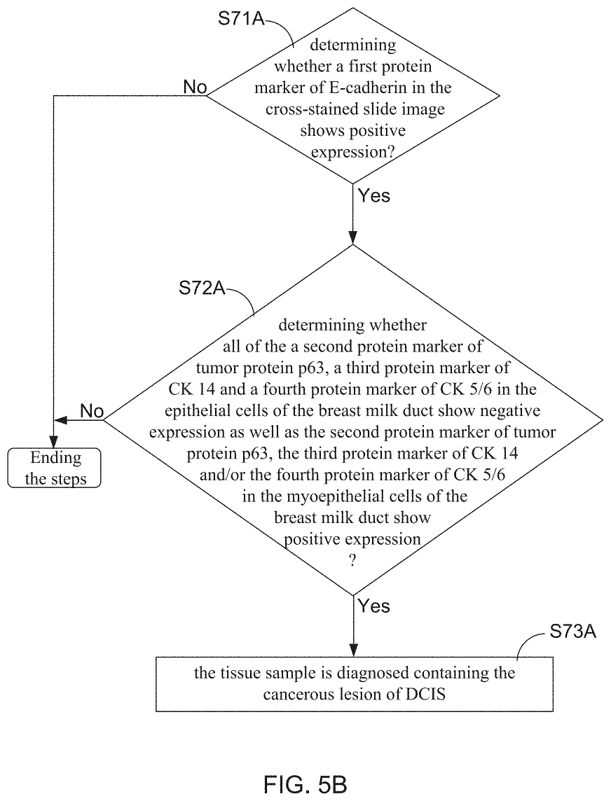

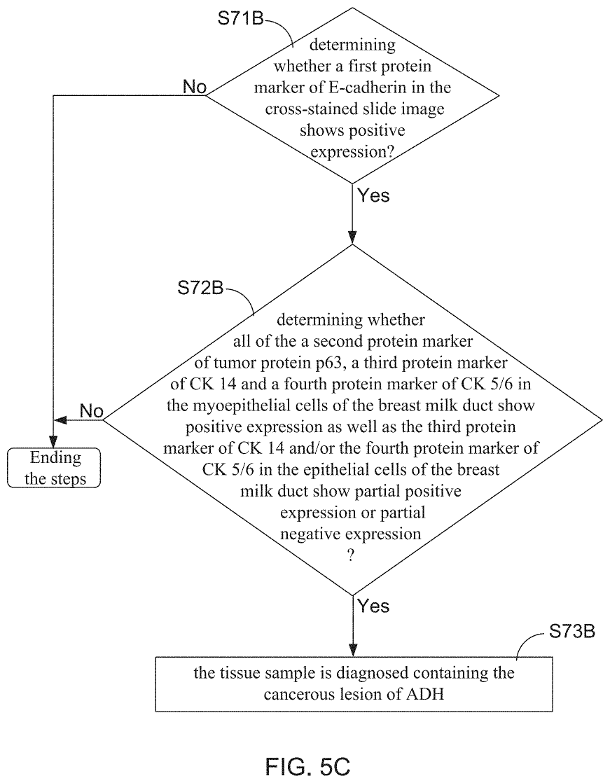

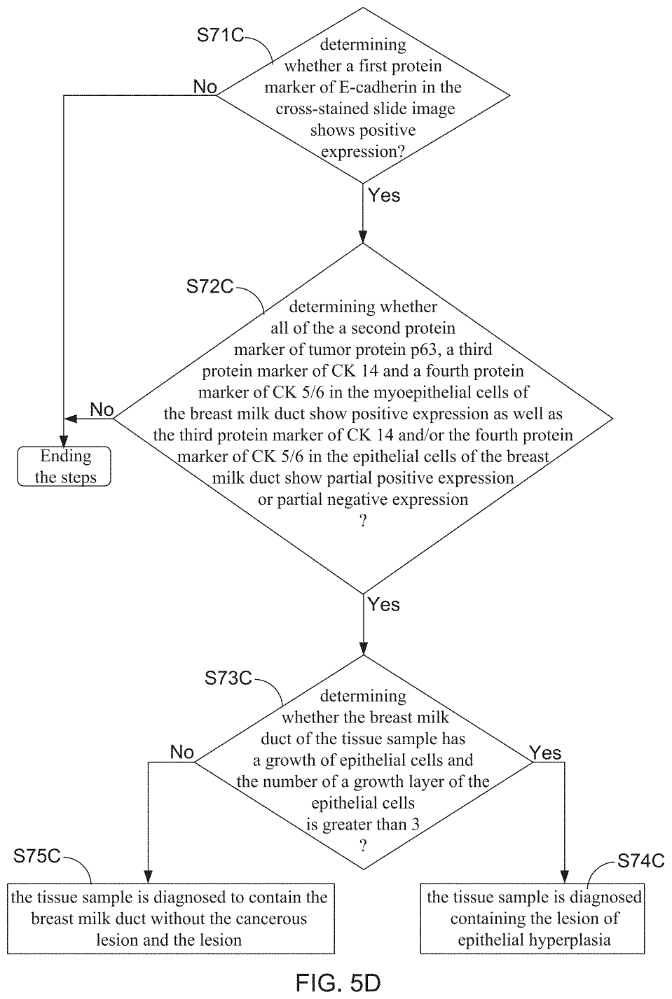

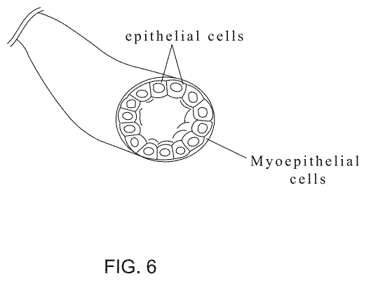

Please refer to FIG. 2B and FIG. 3B again. The cross-staining and multi-biomarker method of the present invention is eventually proceeded to step S7, such that a carcinoma identifying analysis is applied to the plurality of cross-stained slide images. Therefore, identifications of various types of cancerous lesions and/or lesions contained by the tissue sample can be achieved by carrying out image interpretations of the cross-stained slide images. FIG. 5A, FIG. 5B, FIG. 5C, and FIG. 5D show flowchart diagrams for describing detail execution steps of the step S7. In the present invention, step S7 is particularly configured to identify the types of breast cancer from the cross-stained slide images of the tissue sample. For example, FIG. 5A depicts that step S7 is configured to identify the cancerous lesion of basal-like breast carcinoma (BC) from the cross-stained slide images. Before starting to describe the detail steps of step S7, it needs to introduce the basic structure of a breast milk duct's ductal epithelium. FIG. 6 shows a cross-sectional view of the breast milk duct illustrating that the ductal epithelium mainly consisting of luminal epithelial (LEP) cells and myoepithelial (MEP) cells.

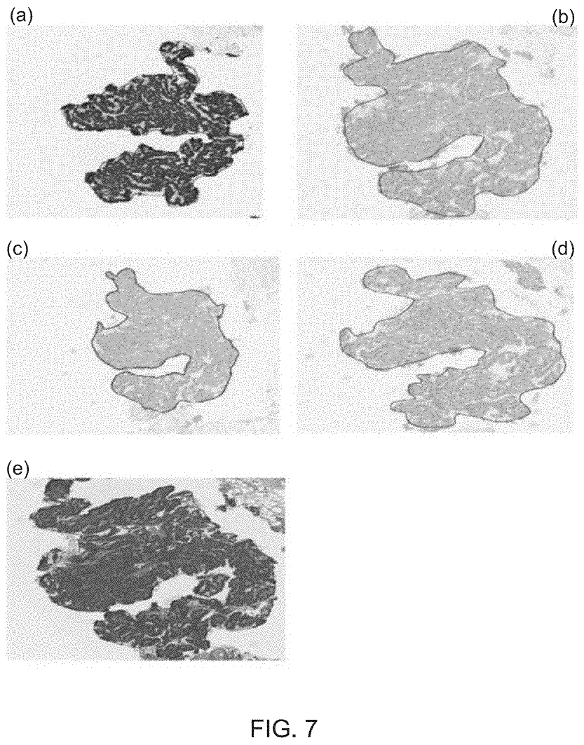

For identifying the cancerous lesion of basal-like breast carcinoma (BC) from the cross-stained slide images, step S71 is designed to determine whether a first protein marker of E-cadherin in the cross-stained slide image shows positive expression or not. Subsequently, step S72 is executed to further determine whether all of the a second protein marker of tumor protein p63, a third protein marker of CK 14 and a fourth protein marker of CK 5/6 in the cross-stained slide image show negative expression or not. In the case of the fact that the determining result of the step S71 and that of step S72 are both “Yes”, the tissue sample is diagnosed containing the cancerous lesion of basal-like breast carcinoma (BC) under the execution of step S73. FIG. 7 shows four frames of cross-stained slide images and one frame of H&E-stained slide image, wherein the four cross-stained slide images comprises a slide image (a) showing the tissue slice with fluorescent labeled E-cadherin, a slide image (b) showing the tissue slice with fluorescent labeled tumor protein p63, a slide image (c) showing the tissue slice with fluorescent labeled CK 14, a slide image (d) showing the tissue slice with fluorescent labeled CK 5/6. Moreover, a slide image (e) displays the tissue slice after being applied with the H&E staining treatment in FIG. 7. By applying image registration and fusion process to the one H&E-stained slide image and the four IHC-stained slide images, four corresponding cross-stained slide images are hence produced for use in the execution of steps S71-S73. Consequently, it is able to identify the cancerous lesion of basal-like breast carcinoma (BC) from the four cross-stained slide images of the tissue sample under the use of the carcinoma identifying analysis.

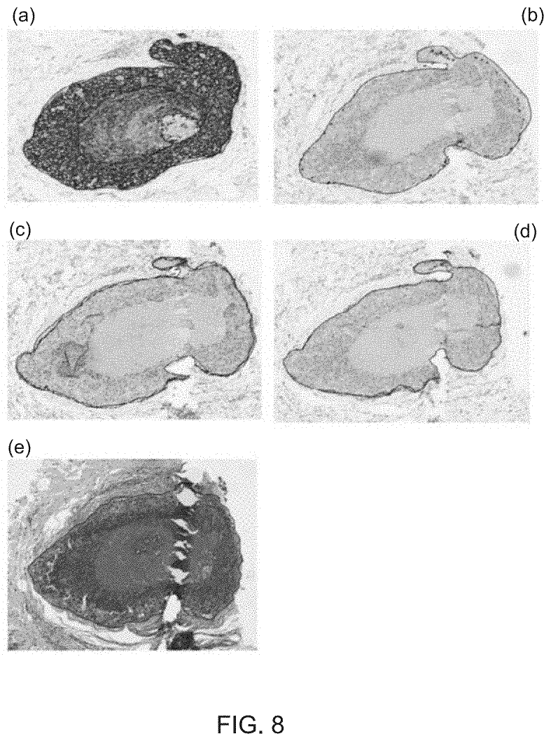

On the other hand, from the FIG. 5B, it is noted that step S7 comprising detail steps of S71A, S72A and S73A are configured to identify the cancerous lesion of duodenal carcinoma in situ (DCIS) from the plurality of cross-stained slide images. In which step S71A is designed to determine whether a first protein marker of E-cadherin in the cross-stained slide image shows positive expression or not. Subsequently, step S72A is executed to further determine whether all of the a second protein marker of tumor protein p63, a third protein marker of CK 14 and a fourth protein marker of CK 5/6 in the epithelial cells of the breast milk duct show negative expression as well as the second protein marker of tumor protein p63, the third protein marker of CK 14 and/or the fourth protein marker of CK 5/6 in the myoepithelial cells of the breast milk duct show positive expression, by carrying out an image interpretation of the cross-stained slide image. In the case of the fact that the determining result of the step S71A and that of step S72A are both “Yes”, the tissue sample is diagnosed containing the cancerous lesion of DCIS) under the execution of step S73A. FIG. 8 shows four frames of cross-stained slide images and one frame of H&E-stained slide image, wherein the four cross-stained slide images comprises a slide image (a) showing the tissue slice with fluorescent labeled E-cadherin, a slide image (b) showing the tissue slice with fluorescent labeled tumor protein p63, a slide image (c) showing the tissue slice with fluorescent labeled CK 14, a slide image (d) showing the tissue slice with fluorescent labeled CK 5/6. Moreover, a slide image (e) displays the tissue slice after being applied with the H&E staining treatment in FIG. 8. By applying image registration and fusion process to the one H&E-stained slide image and the four IHC-stained slide images, four corresponding cross-stained slide images are hence produced for use in the execution of steps S71A-S73A. Consequently, it is able to identify the cancerous lesion of DCIS from the four cross-stained slide images of the tissue sample under the use of the carcinoma identifying analysis.

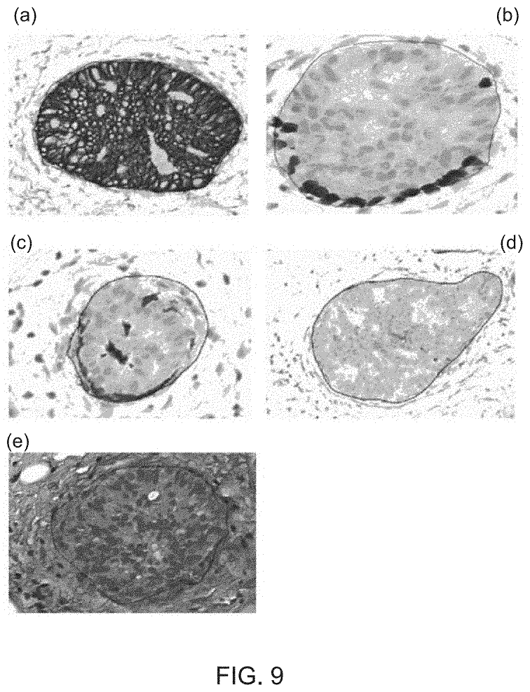

Moreover, FIG. 5C depicts that step S7 comprising detail steps of S71B, S72B and S73B are configured to identify the cancerous lesion of atypical ductal hyperplasia (ADH) from the plurality of cross-stained slide images. In which step S71B is designed to determine whether a first protein marker of E-cadherin in the cross-stained slide image shows positive expression or not. Subsequently, step S72B is executed to further determine whether all of the a second protein marker of tumor protein p63, a third protein marker of CK 14 and a fourth protein marker of CK 5/6 in the myoepithelial cells of the breast milk duct show positive expression as well as the third protein marker of CK 14 and/or the fourth protein marker of CK 5/6 in the epithelial cells of the breast milk duct show partial positive expression or partial negative expression, by carrying out an image interpretation of the cross-stained slide image. When the determining result of the step S71B and that of step S72B are both “Yes”, the tissue sample is diagnosed containing the cancerous lesion of ADH under the execution of step S73B. FIG. 9 shows four frames of cross-stained slide images and one frame of H&E-stained slide image, wherein the four cross-stained slide images comprises a slide image (a) showing the tissue slice with fluorescent labeled E-cadherin, a slide image (b) showing the tissue slice with fluorescent labeled tumor protein p63, a slide image (c) showing the tissue slice with fluorescent labeled CK 14, a slide image (d) showing the tissue slice with fluorescent labeled CK 5/6. Moreover, a slide image (e) shows the tissue slice after being applied with the H&E staining treatment. By applying image registration and fusion process to the one H&E-stained slide image and the four IHC-stained slide images, four corresponding cross-stained slide images are hence produced for use in the execution of steps S71B-S73B. Consequently, it is able to identify the cancerous lesion of ADH from the four cross-stained slide images of the tissue sample under the use of the carcinoma identifying analysis.

Furthermore, FIG. 5D depicts that step S7 comprising detail steps of S71C, S72C, S73C, S74C, and S75C are configured to identify the lesion of epithelial hyperplasia from the plurality of cross-stained slide images. In which step S71C is designed to determine whether a first protein marker of E-cadherin in the cross-stained slide image shows positive expression or not. Subsequently, step S72C is executed to further determine whether all of the a second protein marker of tumor protein p63, a third protein marker of CK 14 and a fourth protein marker of CK 5/6 in the myoepithelial cells of the breast milk duct show positive expression as well as the third protein marker of CK 14 and/or the fourth protein marker of CK 5/6 in the epithelial cells of the breast milk duct show partial positive expression or partial negative expression, by carrying out an image interpretation of the cross-stained slide image. Moreover, step S73C is next executed to determine whether the breast milk duct of the tissue sample has a growth of epithelial cells and the number of a growth layer of the epithelial cells is greater than 3 When all of the determining results of the steps S71B, S72B and S73B are “Yes”, the tissue sample is diagnosed containing the lesion of epithelial hyperplasia under the execution of step S74C. On the contrary, when the determining result of the step S71C and that of step S72C are both “Yes” as well as the determining result of the step S73C is “No”, the tissue sample is diagnosed to contain the breast milk duct without the cancerous lesion and the lesion under the execution of step S75C.

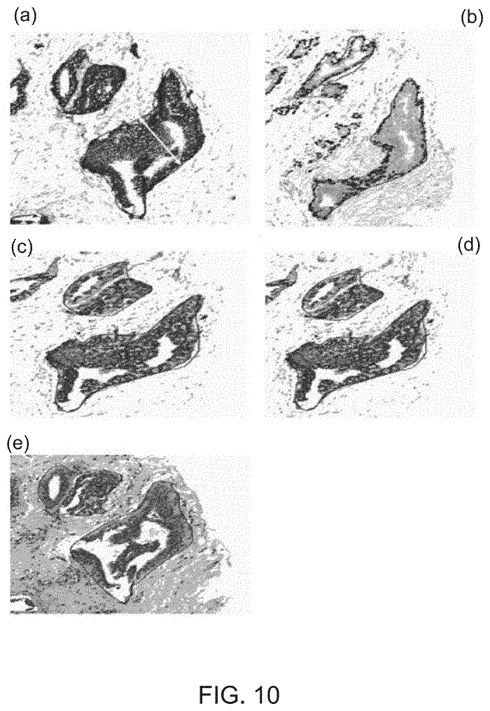

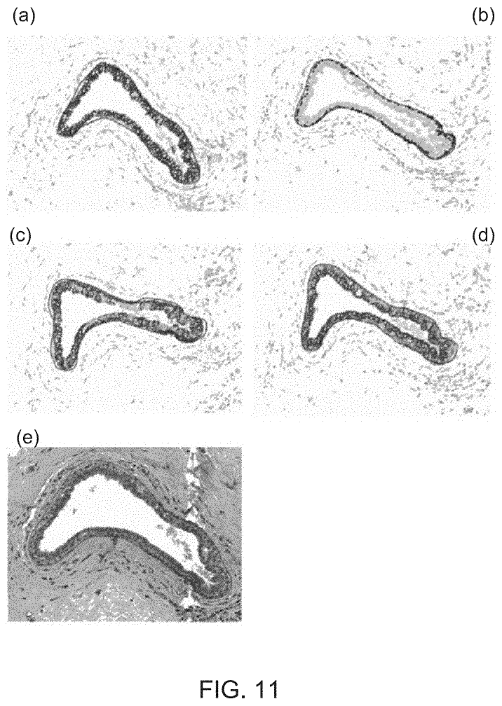

FIG. 10 shows four frames of cross-stained slide images and one frame of H&E-stained slide image, wherein the four cross-stained slide images comprises a slide image (a) showing the tissue slice with fluorescent labeled E-cadherin, a slide image (b) showing the tissue slice with fluorescent labeled tumor protein p63, a slide image (c) showing the tissue slice with fluorescent labeled CK 14, a slide image (d) showing the tissue slice with fluorescent labeled CK 5/6. Moreover, a slide image (e) shows the tissue slice after being applied with the H&E staining treatment in FIG. 10. By applying image registration and fusion process to the one H&E-stained slide image and the four IHC-stained slide images, four corresponding cross-stained slide images are hence produced for use in the execution of steps S71C-S75C. Consequently, it is able to identify the lesion of epithelial hyperplasia from the four cross-stained slide images of the tissue sample under the use of the carcinoma identifying analysis.

In briefly, detailed steps of step S7 particularly designed for completing the identification of various types of cancerous lesions and/or lesions from the cross-stained slide images can be summarized in following Table (1).

TABLE (1)

| Types of | | Expression of protein markers | | | |
|---|---|---|---|---|---|
| cancerous | | Positive: + | | | |
| lesions and/or | Ductal | Negative: − | | | |
| lesions | epithelium | E-Cadherin | P63 | CK14 | CK5/6 |
| Normal | Luminal | + | − | + | + |
| duct | epithelial | | | | |
| organization | (LEP) | | | | |
| | Myoepithelial | + | + | + | + |
| | (MEP) | | | | |
| Epithelial | LEP | + | − | + | + |
| Hyperplasia | MEP | + | + | + | + |
| ADH | LEP | + | − | − | + |
| | | | | | (Partial |
| | | | | | loss) |
| | | | | + | − |
| | | | | (Partial | |
| | | | | loss) | |
| | | | | + | + |
| | | | | (Partial | (Partial |
| | | | | loss) | loss) |
| | MEP | + | + | + | + |
| DCIS | LEP | + | − | − | − |
| | MEP | + | + | + | + |
| BC | LEP | + | − | − | − |
| | MEP | + | − | − | − |

On the other hand, detailed steps of step S7 shown in FIG. 5B can be further summarized in following Table (2), Table (3) and Table (4).

TABLE (2)

| | | Expression of protein markers | | | |
|---|---|---|---|---|---|
| Type of | | Positive: + | | | |
| cancerous | Ductal | Negative: − | | | |
| lesions | epithelium | E-Cadherin | P63 | CK14 | CK5/6 |
| DCIS | LEP | + | − | − | − |
| | MEP | + | + | − | − |

TABLE (3)

| | | Expression of protein markers | | | |
|---|---|---|---|---|---|
| Type of | | Positive: + | | | |
| cancerous | Ductal | Negative: − | | | |
| lesions | epithelium | E-Cadherin | P63 | CK14 | CK5/6 |
| DCIS | LEP | + | − | − | − |
| | MEP | + | − | + | − |

TABLE (4)

| | | Expression of protein markers | | | |
|---|---|---|---|---|---|
| Type of | | Positive: + | | | |
| cancerous | Ductal | Negative: − | | | |
| lesions | epithelium | E-Cadherin | P63 | CK14 | CK5/6 |
| DCIS | LEP | + | − | − | − |
| | MEP | + | − | − | + |

Table (1) also implies that, owing to the fact that some protein markers fail to show full positive expression and/or full negative expression, it is difficult to accurately make a clear distinguishment between the lesion of epithelial hyperplasia and the cancerous lesion of atypical ductal hyperplasia (ADH). In such case, this method categorizes the lesion as ADH either if CK14 or CK5/6 has retained color (partial loss) in epithelial cells.

In other particular case of the fact that protein markers show negative expression in myoepithelial cells but exhibit partial positive expression in epithelial cells, the method categorizes the lesion as basal-like breast carcinoma (BC). On the other hand, since the tissue sample is commonly obtained by using core needle biopsy, it is worth noting that operating error of the core needle biopsy lead the slide images of the tissue slices to have indistinct edges. In such case, as long as the image system installed with the program of this novel method has confirmed that all of the protein markers of tumor protein p63, CK 14 and CK 5/6 in the epithelial cells exhibit negative expression, the tissue sample would be diagnosed containing the cancerous lesion of DCIS by the image system even if the image system fail to simultaneously confirm that the protein marker of tumor protein p63, the protein marker of CK 14 and/or the protein marker of CK 5/6 in the myoepithelial cells show positive expression.

It needs to emphasize that, in spite of FIG. 5A, FIG. 5B, FIG. 5C, FIG. 5D, and FIG. 5D exemplarily showing how to completing the identifications of various breast cancer types by carrying out the image interpretations of the cross-stained slide images with fluorescent labeled E-cadherin, tumor protein p63, CK 14, and CK 5/6, that does not used for becoming limitations of the identification steps for the breast cancer. For example, cytokeratin (CK) 8 or CK 18 can be used for replacing the protein marker of CK 14 for expressing the growth of mesenchymal stem cell of breast milk duct. On the other hand, smooth muscle protein (SMA) can also be used for replacing the protein marker of CK 5/6 for expressing the growth of myoepithelial cells of breast milk duct.

From above descriptions, it is extrapolated that the cross-staining and multi-biomarker method of the present invention can also be applied for assisting in the diagnosis of ovarian cancer, pancreatic cancer, liver cancer, lung cancer, colorectal cancer, stomach cancer, or esophageal cancer. Moreover, in the case of the implementation of this novel method, doctors are able to simultaneously finish a plurality of medical examination items, including: (1) identification and diagnosis of abnormal tumor cells or cancerous lesions, (2) further categorization of the cancerous lesions, (3) accurate histopathologic classification of the abnormal tumor cells, and (4) providing reasonable supports for the cancer treating (or curing) therapy planned and suggested by doctors.

Therefore, through above descriptions, the cross-staining and multi-biomarker method for assisting in cancer diagnosis proposed by the present invention has been introduced completely and clearly; in summary, the present invention includes the advantages of:

(1) The present invention mainly provides a cross-staining and multi-biomarker method for assisting in cancer diagnosis, wherein the method is configured to of firstly divide a plurality of image frames of tissue slices to a group of H&E-stained slide images and a group of IHC-stained slide images. Subsequently, an image registration and fusion process is applied to at least two cross-stained slide images consisting of at least one H&E-stained slide image and at least one IHC-stained slide image, thereby producing a plurality of cross-stained slide images. Consequently, by applying a carcinoma identifying and quantifying analysis to the plurality of cross-stained slide images based on a particularly-designed biomarker expression recognizing flow, various types of cancerous lesions formed in the tissue sample can be effectively detected and eventually diagnosed. Besides, an enrichment ratio of each of the diagnosed cancerous lesions can also be simultaneously calculated. Therefore, it is extrapolated that all types of the abnormal tumor cells or cancerous lesions contained by the tissue sample can be better diagnosed under the implementation of this novel method, without any human-made judgements.

(1) Moreover, this method can be applied to any one type of commercial image registration and cross-image annotation system, such as Leica Biosystems or Vectra imaging system.

The above description is made on embodiments of the present invention. However, the embodiments are not intended to limit scope of the present invention, and all equivalent implementations or alterations within the spirit of the present invention still fall within the scope of the present invention.

What is claimed is:

1. A cross-staining and multi-biomarker method for assisting in cancer diagnosis, comprising steps of:
(1) preparing a tissue sample containing at least one breast milk duct, and then processing the tissue sample to a plurality of tissue slices;
(2) dividing the plurality of tissue slices into a H&E-stained tissue slice group and a IHC-stained tissue slice group, wherein the IHC-stained tissue slice group comprising a first tissue slice group with fluorescent labeled E-cadherin, a second tissue slice group with fluorescent labeled tumor protein p63, a third tissue slice group with fluorescent labeled cytokeratin (CK) 14, and a fourth tissue slice group with fluorescent labeled CK 5/6;
(3) respectively applying a H&E staining treatment and an IHC staining treatment to the tissue slices in the H&E-stained tissue slice group and the tissue slices in the IHC-stained tissue slice group, so as to obtain a plurality of H&E-stained slices and a plurality of IHC-stained slices;
(4) processing the H&E-stained slices to a plurality of H&E-stained slide images, and also processing the IHC-stained slices to a plurality of IHC-stained slide images;
(5) applying an image registration and fusion process to at least two cross-stained slide images consisting of at least one H&E-stained slide image and at least one IHC-stained slide image;
(6) repeating the step (5) until all of the H&E-stained slide images and the IHC-stained slide images have been treated with the image registration and fusion process, thereby producing a plurality of cross-stained slide images; and
(7) applying a carcinoma identifying analysis to the plurality of cross-stained slide images, so as to complete the identification of at least one type of cancerous lesion and/or lesion by carrying out image interpretations of the cross-stained slide images.

2. The method of claim 1, wherein a plurality of protein markers are selected from the tissue slices by the use of a proteomics-based method during the execution of the step (2) and the step (3).

3. The method of claim 2, wherein the protein markers comprising E-cadherin, tumor protein p63, smooth muscle protein (SMA), high molecular weight cytokeratin (HMCK), CK 14, CH 7, CK 5/6, and CK 8/18.

4. The method claim 1, wherein the step (1) comprising following detail steps:
(11) obtaining the tissue sample from the breast milk duct, and then processing the tissue sample to a paraffin block;
(12) sectioning the paraffin block to the plurality of tissue slices; and
(13) applying a fixation process to the tissue slices.

5. The method claim 3, wherein the step (5) comprising following detail steps:
(51) selecting one of the plurality of H&E-stained slide images and at least one of the plurality of IHC-stained slide images, wherein the selected IHC-stained slide image contains at least one protein marker;
(52) applying an image registration process and an image fusion process to the selected H&E-stained slide image and the selected IHC-stained slide image.

6. The method claim 2, wherein the step (7) is configured to identify the cancerous lesion of basal-like breast carcinoma (BC) from the plurality of cross-stained slide images, and comprising following detail steps:
(71) determining whether a first protein marker of E-cadherin in the cross-stained slide image shows positive expression, if yes, proceeding to step (72); otherwise, ending the steps;
(72) determining whether all of the a second protein marker of tumor protein p63, a third protein marker of CK 14 and a fourth protein marker of CK 5/6 in the cross-stained slide image show negative expression, if yes, proceeding to step (73); otherwise, ending the steps; and
(73) the tissue sample is diagnosed containing the cancerous lesion of basal-like breast carcinoma (BC).

7. The method claim 2, wherein the step (7) is configured to identify the cancerous lesion of duodenal carcinoma in situ (DCIS) from the plurality of cross-stained slide images, and comprising following detail steps:
(71A) determining whether a first protein marker of E-cadherin in the cross-stained slide image shows positive expression, if yes, proceeding to step (72A); otherwise, ending the steps;
(72A) determining whether all of the a second protein marker of tumor protein p63, a third protein marker of CK 14 and a fourth protein marker of CK 5/6 in the epithelial cells of the breast milk duct show negative expression as well as the second protein marker of tumor protein p63, the third protein marker of CK 14 and/or the fourth protein marker of CK 5/6 in the myoepithelial cells of the breast milk duct show positive expression, by carrying out an image interpretation of the cross-stained slide images; if yes, proceeding to step (73A); otherwise, ending the steps; and
(73A) the tissue sample is diagnosed containing the cancerous lesion of DCIS.

8. The method claim 2, wherein the step (7) is configured to identify the cancerous lesion of atypical ductal hyperplasia (ADH) from the plurality of cross-stained slide images, and comprising following detail steps:
(71B) determining whether a first protein marker of E-cadherin in the cross-stained slide image shows positive expression, if yes, proceeding to step (72B); otherwise, ending the steps;
(72B) determining whether all of the a second protein marker of tumor protein p63, a third protein marker of CK 14 and a fourth protein marker of CK 5/6 in the myoepithelial cells of the breast milk duct show positive expression as well as the third protein marker of CK 14 and/or the fourth protein marker of CK 5/6 in the epithelial cells of the breast milk duct show partial positive expression or partial negative expression, by carrying out an image interpretation of the cross-stained slide images; if yes, proceeding to step (73B); otherwise, ending the steps; and
(73B) the tissue sample is diagnosed containing the cancerous lesion of ADH.

9. The method claim 2, wherein the step (7) is configured to identify the lesion of epithelial hyperplasia from the plurality of cross-stained slide images, and comprising following detail steps:
(71C) determining whether a first protein marker of E-cadherin in the cross-stained slide image shows positive expression, if yes, proceeding to step (72C); otherwise, ending the steps;
(72C) determining whether all of the a second protein marker of tumor protein p63, a third protein marker of CK 14 and a fourth protein marker of CK 5/6 in the myoepithelial cells of the breast milk duct show positive expression as well as the third protein marker of CK 14 and/or the fourth protein marker of CK 5/6 in the epithelial cells of the breast milk duct show partial positive expression or partial negative expression, by carrying out an image interpretation of the cross-stained slide images; if yes, proceeding to step (73C); otherwise, ending the steps; and
(73C) determining whether the breast milk duct of the tissue sample has a growth of epithelial cells and the number of a growth layer of the epithelial cells is greater than 3; if yes, proceeding to step (74C); otherwise, proceeding to step (75C);
(74C) the tissue sample is diagnosed containing the lesion of epithelial hyperplasia;
(75C) the tissue sample is diagnosed to contain the breast milk duct without the cancerous lesion and the lesion.

10. The method claim 1, being able to be used for assisting in the diagnosis of ovarian cancer, pancreatic cancer, liver cancer, lung cancer, colorectal cancer, stomach cancer, or esophageal cancer.

11. The method claim 1, being able to be applied to any one type of image registration and cross-image annotation systems.
